(19) United States
(12) Patent Application Publication
(10) Pub. No.: US 2014/0180164 A1
(43) Pub. Date: Jun. 26, 2014

(54) TARGETABLE BIOPSY NEEDLE SET AND METHOD OF USING SAME

(71) Applicant: Cook Medical Technologies LLC, Bloomington, IN (US)

(72) Inventor: Thomas Wayne McGhie, Bloomington, IN (US)

(73) Assignee: Cook Medical Technologies LLC, Bloomington, IN (US)

(21) Appl. No.: 13/932,359

(22) Filed: Jul. 1, 2013

Related U.S. Application Data
(60) Provisional application No. 61/740,522, filed on Dec. 21, 2012.

(51) Int. Cl. A61B 10/02

(52) U.S. Cl. CPC A61B 10/0283
USPC 600/566; 600/567

(57) ABSTRACT

A biopsy needle set includes an introducer cannula having an elongate tubular body defining a lumen extending from an open proximal end to an open distal end. The introducer cannula also includes a lateral opening through a distal segment of the elongate tubular body that is in fluid communication with the lumen. The biopsy needle set also includes a biopsy needle configured to be telescopically received within the lumen of the introducer cannula. The biopsy needle includes a preformed curve causing a distal tip of the biopsy needle to point in a direction that is offset from a longitudinal direction of the introducer cannula. The biopsy needle set includes a first extended configuration in which the distal tip is advanced through the lateral opening and a second extended configuration in which the distal tip is advanced through the open distal end.

This application claims priority to provisional patent application 61/740,522, filed on Dec. 21, 2012, with the same title.

### TECHNICAL FIELD

The present disclosure relates generally to a targetable biopsy needle set including an introducer cannula and a biopsy needle, and more particularly to a preformed curve of the biopsy needle facilitating advancement of the biopsy needle through a lateral opening of the introducer cannula.

### BACKGROUND

Various types of biopsy procedures are used to collect one or more tissue samples from a target location within a patient for medical evaluation. Commonly, medical imaging, such as ultrasound, is used to percutaneously guide a biopsy needle to the target location. For example, a clinician may first direct an introducer cannula to the target location and, after the introducer cannula is properly positioned, insert a biopsy needle through the introducer cannula. Various types of biopsy needles exist, including coring type biopsy needles and aspiration type biopsy needles, for collecting tissue samples through the open distal end of the introducer cannula. If the clinician misses the target location or if samples from surrounding tissue are also desired, the clinician may be required to remove the biopsy needle and introducer cannula and reinsert the introducer cannula at a different entry site to being the process again. It should be appreciated that multiple insertions of the introducer cannula may increase patient discomfort and prolong the biopsy procedure.

An exemplary biopsy device is shown in U.S. Pat. No. 5,800,389 to Burney et al. The biopsy device of the Burney et al. reference generally includes an introducer cannula defining a lumen and having a lateral opening in communication with the lumen. The introducer cannula also includes a closed distal tip and a ramp positioned near the closed distal tip that is inclined toward the lateral opening. When an aspiration type biopsy needle is advanced through the introducer cannula, the ramp directs the distal tip of the biopsy needle through the lateral opening. As such, the Burney et al. reference suggests obtaining tissues samples from target areas angularly displaced from a longitudinal axis of the biopsy device. Thus, the Burney et al. reference appears to require placement of the distal tip of the introducer cannula away from a desired tissue sampling location.

The present disclosure is directed toward one or more of the problems or issues set forth above.

### SUMMARY OF THE DISCLOSURE

In one aspect, a biopsy needle set includes an introducer cannula and a biopsy needle. The introducer cannula has an elongate tubular body defining a lumen extending from an open proximal end to an open distal end. The introducer cannula also includes a lateral opening through a distal segment of the elongate tubular body that is in fluid communication with the lumen. The biopsy needle is configured to be telescopically received within the lumen of the introducer cannula and includes a preformed curve. The preformed curve causes a distal tip of the biopsy needle to point in a direction that is offset from a longitudinal direction of the introducer cannula. The biopsy needle set includes a first extended configuration in which the distal tip of the biopsy needle is advanced through the lateral opening of the introducer cannula and a second extended configuration in which the distal tip of the biopsy needle is advanced through the open distal end of the introducer cannula.

In another aspect, a biopsy system includes an introducer set and a coaxial biopsy needle. The introducer set has a distal shearing end and includes an introducer cannula having an elongate tubular body defining a lumen extending from an open proximal end to an open distal end. The introducer cannula also includes a lateral opening through a distal segment of the elongate tubular body that is in fluid communication with the lumen. The coaxial biopsy needle also has a distal shearing end and is configured to be telescopically received within the lumen of the introducer cannula. The coaxial biopsy needle has a preformed curve and includes a needle stylet, which includes a sample collection notch, telescopically received within a needle cannula.

In another aspect, a method of collecting a biopsy sample from a patient using a biopsy needle set includes a step of percutaneously advancing an introducer cannula of the biopsy needle set to a target site within the patient. The method also includes a step of inserting a biopsy needle of the biopsy needle set through a lumen of the introducer cannula. A preformed curve of the biopsy needle is oriented relative to the introducer cannula such that a distal tip of the biopsy needle points toward a lateral opening of the introducer cannula. The distal tip of the biopsy needle is then advanced through the lateral opening of the introducer cannula.

### BRIEF DESCRIPTION OF THE DRAWINGS

FIG. 1 is a partially sectioned side diagrammatic view of a biopsy needle set, according to one embodiment of the present disclosure;

FIG. 2 is a side diagrammatic view of an alternative biopsy needle, according to another embodiment of the present disclosure;

FIG. 3 is a partially sectioned side diagrammatic view of the biopsy needle set of FIG. 1, shown in a first extended configuration;

FIG. 4 is a partially sectioned side diagrammatic view of the biopsy needle set of FIG. 1, shown in a second extended configuration;

FIG. 5 is a partially sectioned side diagrammatic view of the biopsy needle set of the previous Figs. at one stage of a biopsy sample collection procedure;

FIG. 6 is a partially sectioned side diagrammatic view of the biopsy needle set at another stage of a biopsy sample collection procedure;

FIG. 7 is a partially sectioned side diagrammatic view of the biopsy needle set at another stage of a biopsy sample collection procedure;

FIG. 8 is a partially sectioned side diagrammatic view of the biopsy needle set at another stage of a biopsy sample collection procedure;

FIG. 9 is a partially sectioned side diagrammatic view of the biopsy needle set at another stage of a biopsy sample collection procedure;

FIG. 10 is a partially sectioned side diagrammatic view of the biopsy needle set at another stage of a biopsy sample collection procedure; and

FIG. 11 is a partially sectioned side diagrammatic view of the biopsy needle set at another stage of a biopsy sample collection procedure.

### DETAILED DESCRIPTION

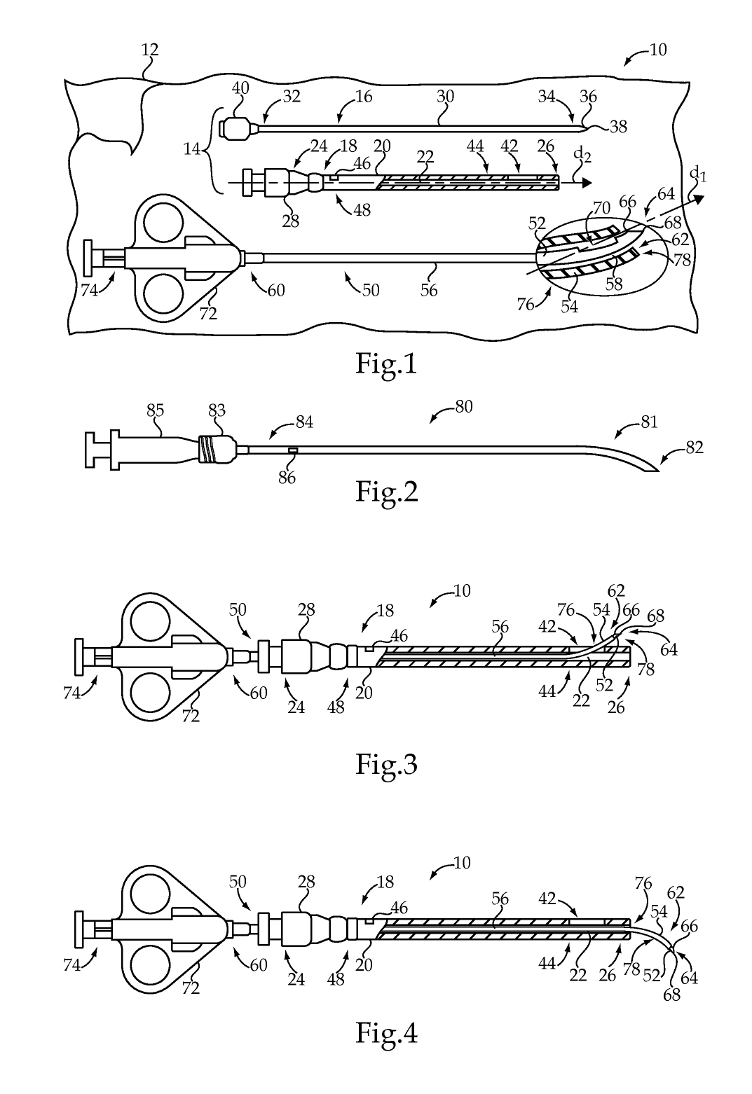

Referring to FIG. 1, there is shown a biopsy needle set 10, or biopsy system, according to one embodiment of the present disclosure. The biopsy needle set 10 may include a number of components, which may be provided within a sterile, tear open package 12, as is known in the art. In performing a biopsy sample collection procedure on a patient, some or all of the components of the biopsy needle set 10 may be used, depending upon the specifics of the procedure to be performed. As should be appreciated, however, the components shown in FIG. 1 might be separately packaged and/or the biopsy needle set 10 might also include components in addition to those shown, including components routinely used in percutaneous vascular procedures.

The biopsy needle set 10 may include an introducer set 14, which may include an introducer stylet 16 configured to be telescopically received within an introducer cannula 18. The introducer cannula 18 may have an elongate tubular body 20 defining a lumen 22 extending from an open proximal end 24 to an open distal end 26. The introducer cannula 18 may also include a handle 28, and/or fitting, disposed at the open proximal end 24 for grasping the introducer cannula 18 and receiving the introducer stylet 16. The introducer stylet 16 may include an elongate body 30 having a proximal end 32 and terminating in a distal shearing end 34. The distal shearing end 34 may include one or more tapered shearing edges 36 terminating in a forward shearing point 38. The introducer stylet 16 may also include a proximal handle 40 for guiding the introducer stylet 16 through the introducer cannula lumen 22 and facilitating proper telescopic positioning of the introducer stylet 16 within the introducer cannula 18. For example, it may be desirable to position the introducer cannula 18 and the introducer stylet 16 such that the distal shearing end of the introducer stylet 16 is advanced through the open distal end 26 of the introducer cannula 18. The components of the introducer set 14 may be made from stainless steel or another suitable material, such as, for example, a metal, metal alloy, or a rigid or semi-rigid plastic.

It should be appreciated that alternative introducer set configurations are also contemplated. For example, the introducer cannula 18 or another component of the biopsy needle set 10 may include a cutting or shearing surface and, as a result, the introducer stylet 16 may be modified or may become unnecessary. In the present disclosure, “proximal” will be used to refer to the end of a component or feature that is closest to a clinician, while “distal” is used to refer to a component or feature that is farthest away from the clinician. Such meanings are consistent with conventional use of the terms and, as such, should be understood by those skilled in the art.

According to the present disclosure, the introducer cannula 18 also includes a lateral opening 42 through a distal segment 44 of the elongate tubular body 20 that is in fluid communication with the lumen 22. The lateral opening 42 may be proximally spaced from the open distal end 26 and may be defined as a location where material has been removed, such as by grinding or laser cutting, or a location that has been formed without material. According to a specific exemplary embodiment, the introducer cannula 18 may be 14-20 gauge and may be approximately 5-15 centimeters in length. The lateral opening 42 may be proximally spaced at least about one centimeter from the open distal end 26 and may have an axial length of at least about one centimeter. Lateral opening 42 may have a rectangular, oval, or other contour that is oblong along the axis of introducer cannula 18. Such dimensions are provided for exemplary purposes only and may vary depending on the particular application. For reasons that will become more apparent below, an indicator 46 corresponding to the lateral opening 42 may be provided along a proximal segment 48 of the introducer cannula 18, which may include all or portions of the handle 28.

The biopsy needle set 10 may also include a biopsy needle 50 that is configured to be telescopically received within the lumen 22 of the introducer cannula 18. According to the exemplary embodiment, the biopsy needle 50 may be a coaxial biopsy needle and may include a needle stylet 52 telescopically received within a needle cannula 54 (shown at enlarged distal end of biopsy needle 50). The needle cannula 54 may include an elongate tubular body 56 defining a lumen 58 extending from an open proximal end 60 to an open distal end 62. The needle stylet 52 may include a distal shearing end 64 including one or more tapered shearing edges 66 terminating in a forward shearing point 68. However, alternative cutting surfaces are also contemplated. The needle stylet 52 also includes a sample collection notch 70 positioned near the distal shearing end 64 for collecting tissue samples in a known manner. The sample collection notch 70 may be defined as a location where material has been removed, such as by grinding or laser cutting, or a location that has been formed without material. According to a specific exemplary embodiment, the biopsy needle 50 may be 16-22 gauge, defining an outer diameter sized to be received within the introducer cannula 18, and may be approximately 10-20 centimeters in length. The sample collection notch 70 may have a throw, or axial length, of approximately 10-20 millimeters.

The biopsy needle 50 may also include a proximal handle 72 for moving the needle stylet 16 and the needle cannula 18 relative to one another. For example, the needle cannula 18 and the needle stylet 16 may be arranged such that the needle cannula 18 is distally advanced over the sample collection notch 70 of the needle stylet 16. In use, the proximal handle 72 may facilitate forward advancement of the needle stylet 16 to expose the sample collection notch 70 and, thereafter, forward movement of the needle cannula 18 to cut out a specimen of tissue that has prolapsed into the sample collection notch 70. According to some embodiments, the proximal handle 72 may include a spring-loaded mechanism 74 to advance the needle cannula 18 over the needle stylet 16 very quickly to prevent the prolapsed tissue in the sample collection notch 70 from being displaced as the needle cannula 18 advances over the needle stylet 16.

According to the present disclosure, the biopsy needle 50 also includes a preformed curve 76 that causes a distal tip 78 of the biopsy needle 50 to point in a direction d₁ that is offset from a longitudinal axis, or direction, d₂ of the introducer cannula 18. The components of the biopsy needle 50 may be stainless steel or may be constructed from any other suitable material including, but not limited to, metals, metal alloys such as nickel titanium alloys known to be shape memory metals, and rigid or semi-rigid plastics. The distal curvature, or preformed curve 76, of the biopsy needle 50 may be formed during manufacturing or may be formed by the clinician prior to a procedure to impose a desired curvature.

As an alternative to the coring type biopsy needle 50 described above, the biopsy needle set 10 may include an aspiration type biopsy needle, shown at 80 in FIG. 2. The aspiration type biopsy needle 80 may be a hollow needle configured to be telescopically received within the lumen 22 of the introducer cannula 18. The aspiration type biopsy needle 80 also includes a preformed distal curve 81, similar to the preformed curve 76 described above, and may have a blunt distal tip or may terminate in a sharp distal point 82. A hub 83, such as a luer lock fitting, may be provided at a proximal end 84 of the aspiration type biopsy needle 80 to facilitate attachment of a syringe 85 or vacuum device. An indicator 86, similar to indicator 86 described above, which corresponds to the direction of the preformed distal curve 81 may be provided at the proximal end 84 of the aspiration type biopsy needle 80 and may be useful in orienting the aspiration type biopsy needle 80 relative to the introducer cannula 18 during a procedure.

The biopsy needle set 10 should not be limited to the above described coring type biopsy needle 50 or aspiration type biopsy needle 80. Alternative biopsy needles, incorporating a preformed curve, may also be used. According to an additional example, a biopsy needle for use with the biopsy needle set 10 may include a spirally shaped tissue-receiving element, which may be received within and used in combination with a catheter-like tubular element. Spirally shaped tissue-receiving elements for taking tissue samples are known and may be modified for use with the biopsy needle set 10 to include a preformed curve, as described herein.

With reference again to the embodiment of FIG. 1, the biopsy needle set 10 includes a first extended configuration in which the distal tip 78 of the biopsy needle 50 is advanced through the lateral opening 42 of the introducer cannula 18, as shown in FIG. 3. As will be described below, a clinician may achieve the first extended configuration by orienting, or rotating, the biopsy needle 50, during advancement, such that the distal tip 78 of the biopsy needle 50 is aligned with or points toward the lateral opening 42. The biopsy needle set 10 also includes a second extended configuration in which the distal tip 78 of the biopsy needle 50 is advanced through the open distal end 26 of the introducer cannula 18, as shown in FIG. 4. The clinician may achieve the second extended configuration by orientating the biopsy needle 50 such that the distal tip 78 of the biopsy needle 50 is not aligned with the lateral opening 42. The indicator 46 on the proximal segment 48 of the introducer cannula 18, which corresponds to the lateral opening 42, along with any markings on the biopsy needle 50 corresponding to the preformed curve 76, may assist in orienting the biopsy needle 50 as desired.

### INDUSTRIAL APPLICABILITY

The present disclosure is generally applicable to biopsy needles. More specifically, the present disclosure is applicable to various types of biopsy needles, including coring type biopsy needles and aspiration type biopsy needles used in percutaneous biopsy sample collection procedures. Yet further, the present disclosure is applicable to targetable biopsy needles useful in fine-tuning the position of the biopsy needle relative to an introducer set and useful in collecting samples from a wider tissue sample collection area based on only one initial insertion of the introducer set.

Referring generally to FIGS. 1-11, an exemplary embodiment of a biopsy needle set 10 according to the present disclosure generally includes an introducer set 14 and a biopsy needle 50. The introducer set 14 may include an introducer stylet 16 configured to be telescopically received within an introducer cannula 18. The introducer cannula 18 may have an elongate tubular body 20 defining a lumen 22 extending from an open proximal end 24 to an open distal end 26. The introducer stylet 16 may include an elongate body 30 having a proximal end 32 and terminating in a distal shearing end 34. The introducer cannula 18 also includes a lateral opening 42 through a distal segment 44 of the elongate tubular body 20 that is in fluid communication with the lumen 22.

The biopsy needle 50, which may be a coaxial biopsy needle, is configured to be telescopically received within the lumen 22 of the introducer cannula 18. According to the exemplary embodiment, the biopsy needle 50 may include a needle stylet 52 telescopically received within a needle cannula 54. The needle cannula 54 may include an elongate tubular body 56 defining a lumen 58 extending from an open proximal end 60 to an open distal end 62. The needle stylet 52 may include a distal shearing end 64 including one or more tapered shearing edges 66 terminating in a forward shearing point 68. The needle stylet 52 also includes a sample collection notch 70 positioned near the distal shearing end 64 for collecting tissue samples. The biopsy needle 50 also includes a preformed curve 76 that causes a distal tip 78 of the biopsy needle 50 to point in a direction d₁ that is offset from a longitudinal axis, or direction, d₂ of the introducer cannula 18. The biopsy needle 50 may also include a proximal handle 72 for moving the needle stylet 52 and the needle cannula 54 relative to one another, as described above.

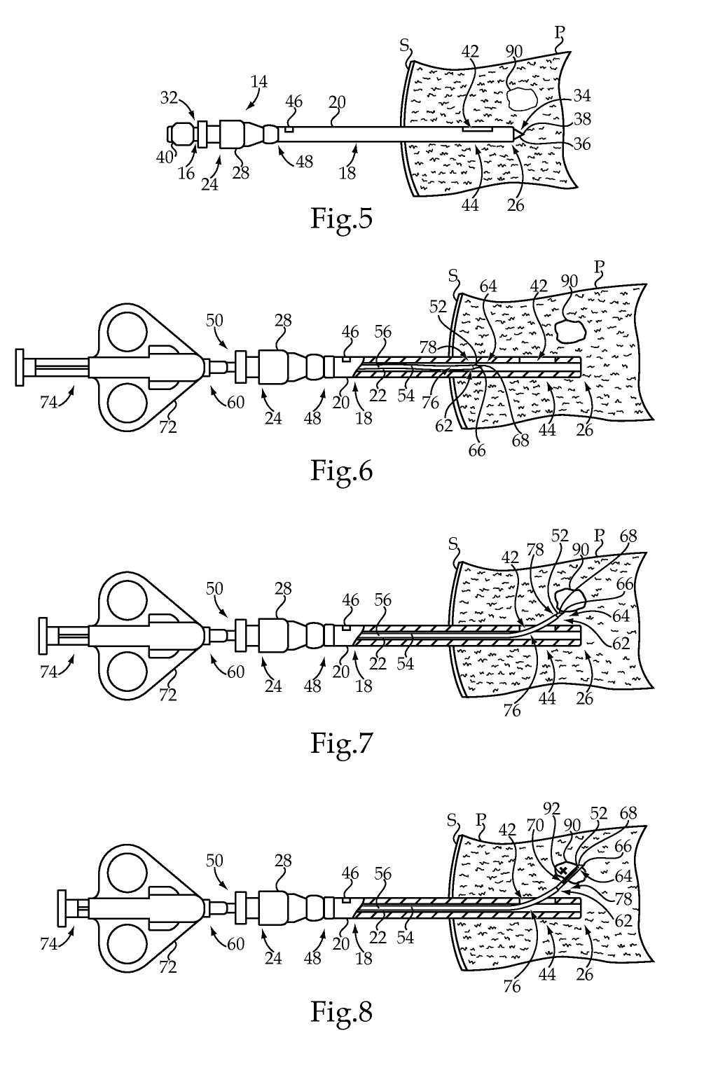

With particular reference to FIG. 5, a percutaneous biopsy sample collection procedure using the biopsy needle set 10 disclosed herein will be described with reference to a target site 90 within a patient P. The target site 90 may represent an area of interest from which a biopsy sample is desired. Prior to the procedure, a clinician may initialize the biopsy needle 50 by grasping the handle 72 and proximally retracting the spring-loaded mechanism 74. After the biopsy needle 50 is initialized, and at a first stage of the procedure, the clinician may position the introducer set 14 through the skin S of the patient P to gain access to the target site 90. For example, the clinician may use imaging guidance to advance the introducer set 14, with the distal shearing end 34 of the introducer stylet 16 distally advanced beyond the open distal end 26 of the introducer cannula 18, toward the target site 90.

The clinician may attempt to position the open distal end 26 of the introducer cannula 18 as close as possible to the target site 90. However, as shown in FIG. 5, the positioning may require fine-tuning According to conventional biopsy sample collection procedures, the inaccurate positioning of the introducer set 14 may require the clinician to remove and reinsert the introducer set 14, resulting in an additional entry site through the skin S. However, utilizing the biopsy needle set 10 of the present disclosure, subsequent insertions may be avoided.

With the introducer cannula 18 still positioned near the target site 90, the introducer stylet 16 may be removed from the introducer cannula 18 and replaced with the biopsy needle 50, as shown in FIG. 6. Before or after insertion of the biopsy needle 50, the clinician may rotate the introducer cannula 18 such that the lateral opening 42 is facing the target site 90, such as with the assistance of the lateral opening indicator 46 positioned along the proximal segment 48 of the introducer cannula 18. With the lateral opening 42 properly positioned, the preformed curve 76 of the biopsy needle 50 may be oriented relative to the introducer cannula 18 such that the distal tip 78 of the biopsy needle 50 points toward, or is otherwise substantially aligned with, the lateral opening 42 of the introducer cannula 18. With the lateral opening 42 and preformed curve 76 properly aligned, the distal tip 78 of the biopsy needle 50 may be advanced through the lateral opening 42 of the introducer cannula 18, as shown in FIG. 7. As should be appreciated, the particular curvature of the preformed curve 76, along with the position and dimensions of the lateral opening 42 may be arrived at through testing to determine desired results.

Turning now to FIG. 8, to collect a first biopsy sample from a first target location 92, which may be located within the target area 90, the handle 72 or, more specifically, the spring-loaded mechanism 74 may be pushed in a distal direction to advance the needle stylet 52 distally beyond the needle cannula 54 to expose the sample collection notch 70. This permits prolapsed tissue to collect in the sample collection notch 70 from the first target location 92. The clinician then continues to distally advance the handle 72 to distally advance the needle cannula 54 over the needle stylet 52. According to the exemplary embodiment, the spring-loaded mechanism 74 advances the needle cannula 54 over the needle stylet 52 very quickly in order to prevent the prolapsed tissue in the sample collection notch 70 from being displaced as the needle cannula 54 advances over the needle stylet 52.

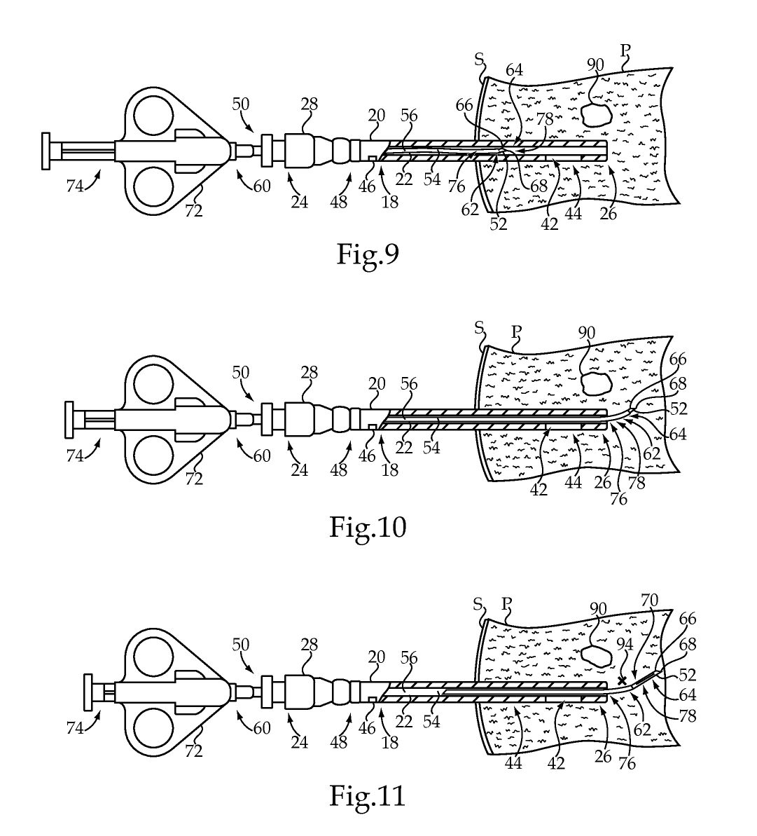

The biopsy needle 50 may be subsequently withdrawn from the introducer cannula 18 and the first biopsy sample 92 may be removed. With the introducer cannula 18 still positioned as shown, the biopsy needle 50 may be reinserted through the lumen 22 of the introducer cannula 18, as shown in FIG. 9. To collect a second biopsy sample from an area spaced from the target area 90, the preformed curve 76 of the biopsy needle 50 may be oriented relative to the introducer cannula 18 such that the distal tip 78 of the biopsy needle 50 points away from, or is not aligned with, the lateral opening 42 of the introducer cannula 18. The distal tip 78 of the biopsy needle 50 may then be advanced through the open distal end 26 of the introducer cannula 18, as shown in FIG. 10. The needle stylet 52 may be moved relative to the needle cannula 54, in a manner described above, to expose the sample collection notch 70 and receive a second biopsy sample from a second target location 94 within the sample collection notch 70, as shown in FIG. 11.

It should be appreciated that the introducer cannula 18 may be rotated and/or axially advanced or withdrawn to reposition one or both of the open distal end 26 and the lateral opening 42 to provide a wide sample collection area that requires only one initial insertion of the introducer set 14. The biopsy needle set 10 disclosed herein provides the flexibility to function as a conventional biopsy needle set, collecting tissue samples through the open distal end 26, and/or collect tissue samples through the lateral opening 42 after properly aligning the preformed curve 76 of the biopsy needle 50 with the lateral opening 42. The flexibility may be particularly useful when fine-tuning of the biopsy needle positioning is needed and/or when samples are required from a wide sample collection area.

It should be understood that the above description is intended for illustrative purposes only, and is not intended to limit the scope of the present disclosure in any way. Thus, those skilled in the art will appreciate that other aspects of the disclosure can be obtained from a study of the drawings, the disclosure and the appended claims.

What is claimed is:

1. A biopsy needle set, comprising:
an introducer cannula having an elongate tubular body defining a lumen extending from an open proximal end to an open distal end, wherein the introducer cannula also includes a lateral opening through a distal segment of the elongate tubular body that is in fluid communication with the lumen; and
a biopsy needle configured to be telescopically received within the lumen of the introducer cannula and including a preformed curve, wherein the preformed curve causes a distal tip of the biopsy needle to point in a direction that is offset from a longitudinal direction of the introducer cannula;
wherein the biopsy needle set includes a first extended configuration in which the distal tip of the biopsy needle is advanced through the lateral opening of the introducer cannula and a second extended configuration in which the distal tip of the biopsy needle is advanced through the open distal end of the introducer cannula.

2. The biopsy needle set of claim 1, wherein the biopsy needle includes a needle stylet telescopically received within a needle cannula, wherein the needle stylet includes a sample collection notch.

3. The biopsy needle set of claim 2, further including a handle attached at a proximal end of the biopsy needle, wherein the handle includes a spring loaded mechanism configured to move the needle stylet and the needle cannula relative to one another.

4. The biopsy needle set of claim 2, wherein at least one of the needle stylet and the needle cannula includes a distal shearing end.

5. The biopsy needle set of claim 1, wherein the biopsy needle is an aspiration type biopsy needle including a hub attached at a proximal end thereof, wherein the hub is configured for attachment with a syringe or a vacuum device.

6. The biopsy needle set of claim 1, further including an introducer stylet having a distal shearing end and configured to be telescopically received within the introducer cannula.

7. The biopsy needle set of claim 1, wherein the lateral opening is proximally spaced at least about one centimeter from the open distal end.

8. The biopsy system of claim 1, wherein the lateral opening has an axial length of at least about one centimeter.

9. The biopsy system of claim 1, further including an indicator corresponding to the lateral opening positioned along a proximal segment of the introducer cannula.

10. A biopsy system, comprising:
an introducer set having a distal shearing end and including an introducer cannula having an elongate tubular body defining a lumen extending from an open proximal end to an open distal end, wherein the introducer cannula also includes a lateral opening through a distal segment of the elongate tubular body that is in fluid communication with the lumen; and
a coaxial biopsy needle having a distal shearing end, wherein the coaxial biopsy needle is configured to be telescopically received within the lumen of the introducer cannula and includes a preformed curve, wherein the coaxial biopsy needle includes a needle stylet telescopically received within a needle cannula, wherein the needle stylet includes a sample collection notch.

11. The biopsy system of claim 10, further including an introducer stylet configured to be telescopically received within the introducer cannula and having the distal shearing end of the introducer set.

12. The biopsy system of claim 10, further including a handle attached at a proximal end of the coaxial biopsy needle.

13. The biopsy system of claim 12, wherein the handle includes a spring loaded mechanism configured to move the needle stylet and the needle cannula relative to one another.

14. The biopsy system of claim 10, wherein the lateral opening is proximally spaced at least about one centimeter from the open distal end.

15. The biopsy system of claim 10, wherein the lateral opening has an axial length of at least about one centimeter.

16. The biopsy system of claim 10, further including an indicator corresponding to the lateral opening positioned along a proximal segment of the introducer cannula.

17. A method of collecting a biopsy sample from a patient using a biopsy needle set, the biopsy needle set including: an introducer cannula having an elongate tubular body defining a lumen extending from an open proximal end to an open distal end, wherein the introducer cannula also includes a lateral opening through a distal segment of the elongate tubular body that is in fluid communication with the lumen; and a biopsy needle configured to be telescopically received within the lumen of the introducer cannula and including a preformed curve, wherein the preformed curve causes a distal tip of the biopsy needle to point in a direction that is offset from a longitudinal direction of the introducer cannula, the method comprising the steps of:
percutaneously advancing the introducer cannula to a target site within the patient;
inserting the biopsy needle through the lumen of the introducer cannula;
orienting the preformed curve of the biopsy needle relative to the introducer cannula such that the distal tip of the biopsy needle points toward the lateral opening of the introducer cannula; and
advancing the distal tip of the biopsy needle through the lateral opening of the introducer cannula.

18. The method of claim 17, further including:
telescopically moving a needle stylet of the biopsy needle relative to a needle cannula of the biopsy needle after the advancing step to expose a sample collection notch of the needle stylet; and
receiving a first biopsy sample from a first target location within the sample collection notch.

19. The method of claim 18, further including:
inserting the biopsy needle through the lumen of the introducer cannula;
orienting the preformed curve of the biopsy needle relative to the introducer cannula such that the distal tip of the biopsy needle points away from the lateral opening of the introducer cannula; and
advancing the distal tip of the biopsy needle through the open distal end of the introducer cannula.

20. The method of claim 19, further including:
telescopically moving the needle stylet relative to the needle cannula after advancing the distal tip of the biopsy needle through the open distal tip of the introducer cannula to expose the sample collection notch; and
receiving a second biopsy sample from a second target location within the sample collection notch.
